(19) United States
(12) Patent Application Publication
(10) Pub. No.: US 2014/0039565 A1
(43) Pub. Date: Feb. 6, 2014

(54) SYSTEMS AND METHODS FOR INJECTING FLUID INTO BONE AND FOR INSERTING BONE SCREWS AND BONE SCREWS FOR SAME

(71) Applicants: Paul A. Martineau, Westmount (CA); Louis-Philippe Lefebvre, Montreal (CA)

(72) Inventors: Paul A. Martineau, Westmount (CA); Louis-Philippe Lefebvre, Montreal (CA)

(21) Appl. No.: 13/985,153

(22) PCT Filed: Feb. 14, 2012
(86) PCT No.: PCT/CA12/50082

§ 371 Date: Oct. 21, 2013

Related U.S. Application Data
(60) Provisional application No. 61/442,366, filed on Feb. 14, 2011.
(60) Provisional application No. 61/442,376, filed on Feb. 14, 2011.

(51) Int. Cl. A61B 17/86

(52) U.S. Cl. CPC A61B 17/864
USPC 606/304

(57) ABSTRACT

Systems and methods for the insertion of bone screws and for fluid injection using bone screws are described. A bone screw used for fluid injection includes a screw body with a central passage extending at least partially into the screw body and defining an inlet end which includes an opening configured to matingly receive therein an injector tip of a fluid injector. The screw body includes a number of fluid flow passages extending through the screw body wall such as to direct fluid in an outward direction from within the central passage, through the screw body, and into the tissue site surrounding the screw. Another bone screw which is inserted using a guide wire includes a cannulated passage having a non-circular cross-section configured to rotationally interconnect the guide wire such that relative axial rotation between the guide wire and the bone screw is prevented.

### CROSS REFERENCE TO RELATED APPLICATIONS

The present application claims the benefit of U.S. Provisional Patent Application Ser. No. 61/442,376 filed Feb. 14, 2011 and U.S. Provisional Patent Application Ser. No. 61/442,366 filed Feb. 14, 2011, the entire disclosure of each of which is incorporated herein by reference.

### TECHNICAL FIELD

The present disclosure relates generally to bone screws, and more particularly to systems and methods for inserting such bone screws and/or for injecting fluid into bones.

### BACKGROUND

Biomedical screws such as bone screws are commonly used, for example, to join together fractured fragments of a broken bone. Such bone screws are inserted in place within the bone or other tissue during a surgical intervention to precisely locate the screw in a desired location. In the case of fracture fixation, the bone screw is often inserted such that it straddles the fracture site and stabilizes the fractured fragments together. Compression bone screws draw the fractured bone segments closer together, which favours prompt healing while decreasing the risk of non-union. Dense metals, mostly stainless steel and titanium, have been traditionally used for the production of such bone screws.

As the gap between fractured surfaces has an important impact on union of fractured fragments of bone together, reduction of the gap is particularly important to increase union rates and reduce the healing period. Accurate placement of the screw, minimization of the space between fracture surfaces and promoting faster healing are examples of areas where improvement is sought in this respect. Thus, there is an interest to improve the way these screws are inserted, such as to minimize the risk of failure of the screw during the surgery.

Guide wires are typically used to aid with the insertion of such bone screws into the bone or other tissue, and are particularly recommended in minimally invasive surgeries where they help the insertion of the screws or other devices to be inserted. Such guide wires are most commonly composed of very thin wire having a circular cross-section, such that cannulated bone screws can be slid onto and along the guide wire. Typically, a guide wire is carefully inserted through the soft tissue and into the bone site to be reinforced by the bone screw, with the guide wire being disposed in a position and orientation corresponding to that desired for the bone screw, such that the axis defined by the guide wire corresponds to a desired insertion axis along which the screw is to be positioned when inserted into the bone.

These known wires have circular cross sections and are only used to guide the insertion or the positioning of the tissues where the screw is inserted. Accordingly, the screw is allowed to rotate freely around the guide wire during the surgical procedure. Once such a guide wire is positioned in place, the cannulated bone screw is slid onto and along the guide wire until the bone screw is positioned on the surface of the bone into which it is to be driven. The bone screw is then driven into the bone using a suitable screw driver.

As noted above, the gap between fractured surfaces has an important impact on union of fractured fragments of bone together. Different material compounds have been used to attempt to reduce the fracture gaps, reduce micro-movement and increase the union rates of fractured bones. Bone grafts have been extensively used to fill fracture gaps and promote fusion. Bone graft substitutes have also been developed and used as alternative to natural bone grafts. They can be produced with natural materials (demineralised bone matrix and coral for example) or synthetic materials (calcium phosphate, hydroxyapatite, bioglass for example). Bone growth factors such as bone morphogenetic proteins (BMPs) have also been developed and used to promote bone repair.

However, regardless of the material chosen which is to be injected into the bone for the purposes of promoting healing thereof, it typically remains difficult to access the fracture site or other site within the bone itself into which the material is to be inserted. Therefore the accurate injection of such material to the desired bone site can be challenging.

Accordingly, there remains a need for an improved device, system and/or method for inserting biomedical screws and injecting material into a bone and/or soft tissue site in a manner which limits the invasiveness of the procedure and nevertheless enables accurate placement of the injected material.

### SUMMARY

There is provided a system for injecting fluid into a region of a bone comprising: a fluid injector including a syringe having a storage reservoir for the fluid and an injector tip from which the fluid is ejected when the syringe is actuated; and a bone screw comprising a screw body and having at least one external thread thereon, a bore extending at least partially into the screw body and defining an inlet end through which fluid from the fluid injector is received when the fluid injection and bone screw are connected in fluid flow communication, and one or more fluid ejection passages being defined in the screw body and extending from the bore to an outer surface of the screw body, such as to provide fluid flow in an outward direction from within the bore to the region of the bone surrounding the bone screw when said bone screw is inserted into said bone region and fluid is injected into the bore of the bone screw by the fluid injector.

There is also provided a kit for injecting fluid into a tissue site comprising: a fluid injector including a storage reservoir for the fluid and an injector tip from which the fluid is ejected when the syringe is actuated; and an orthopaedic screw comprising a screw body and having at least one external thread on an outer surface thereof, a bore extending at least partially into the screw body and defining an inlet end which includes an adapter opening configured to matingly receive therein the injector tip of the fluid injector, the screw body having an annular portion which surrounds the bore therein and has a radial wall thickness, and one or more fluid ejection passages being defined in at least the annular portion of the screw body and providing fluid flow communication between the bore and the outer surface of the screw body such as to direct fluid in an outward direction from within the bore, through the screw body, and into the tissue site surrounding the orthopaedic screw when the screw is inserted therein and the fluid is injected into the bore of the orthopaedic screw by the fluid injector.

There is also provided a fluid injecting device for injecting fluid into a bone using a fluid injector including an injector tip from which the fluid is ejected when the fluid injector is actuated, the fluid injecting device comprising a bone screw having a screw body and having at least one external thread thereon, a bore extending at least partially into the screw body and defining an inlet end which is configured to matingly receive the injector tip of the fluid injector therein, and one or more fluid ejection passages being defined in the screw body and extending from the bore to an outer surface of the screw body, such as to provide fluid flow in an outward direction from within the bore to the region of the bone surrounding the bone screw when said bone screw is inserted into said bone region and fluid is injected into the bore of the bone screw by the fluid injector.

There is also provided a method of injecting a bone repair promoting material into a bone, comprising: inserting a bone screw into the bone, the bone screw having a bore extending at least partially into a screw body which includes an annular portion around said bore, an adapter opening being formed in an end of the screw body and opening into the bore, and one or more fluid flow passages extending through the annular portion from the bore to an outer surface of the screw body; connecting an injector and the bone screw in fluid flow communication by mating an injector tip of the injector with the adapter opening in the screw body, the injector having a reservoir for the bone repair promoting material and a needle portion having one end connected to the reservoir and a remote end defining the injector tip; and injecting the bone repair promoting material into the bone by actuating the injector to force said material from the reservoir, through the needle and into the bore of the bone screw, the material thereby flowing through the fluid flow passages extending through the annular portion of the screw body and into the bone surrounding the bone screw.

There is further provided a system for inserting a biomedical screw comprising: a guide wire having a longitudinal axis and defining a non-circular cross-sectional shape; and the screw including a screw body having a bore extending therethrough and defining a cannula for receiving the guide wire, the cannula having a non-circular cross-section configured to receive the guide wire therein and rotationally interconnect the guide wire and the screw together, such that relative axial rotation between the guide wire and the screw is prevented.

There is further provided a guide wire for use in insertion of a bone screw, the guide wire comprising an elongated wire body having a central longitudinal axis and an outer perimeter, the elongated wire body having a cross-sectional shape in a plane substantially perpendicular to the central longitudinal axis, the cross-sectional shape being non-circular.

There is further provided a bone screw for use with such a guide wire, the bone screw comprising a screw body having a bore extending therethrough and defining a cannula for receiving the guide wire, the cannula having a non-circular cross-section configured to receive the guide wire therein and rotationally interconnect the guide wire and the screw together, such that relative axial rotation between the guide wire and the screw is prevented.

There is further provided a kit for inserting a biomedical screw comprising: a guide wire having a longitudinal axis and defining a non-circular cross-sectional shape; the screw including a screw body having a bore extending therethrough and defining a cannula for receiving the guide wire, the cannula having a non-circular cross-section configured to receive the guide wire therein and rotationally interconnect the guide wire and the screw together, such that relative axial rotation between the guide wire and the screw is prevented; and a drive element adapted to engage the guide wire for rotation of the guide wire about the longitudinal axis thereof, thereby also rotating the screw when the screw and the guide wire are interconnected.

There is further provided use of a guide wire to insert a bone screw having a screw cannula extending therethrough, the guide wire and the screw cannula both having a non-circular cross-sectional shape, comprising mating the guide wire and the screw cannula to rotationally interconnect the guide wire and bone screw such that relative rotation therebetween is prevented, and rotating the guide wire on which the bone screw is disposed, thereby rotating the bone screw.

There is further provided a method of inserting a bone screw into a tissue site, comprising: positioning a proximal end of a guide wire into the tissue site at a desired location for insertion of the bone screw, the guide wire defining a non-circular transverse cross-section; inserting a cannulated bone screw onto a distal end of the guide wire and sliding the bone screw along the guide wire to the desired location, the bone screw having a non-circular cannula corresponding in cross-sectional shape to the transverse cross-section of the guide wire; inserting a drive handle onto the distal end of the guide wire, the driving handle having a bore therein which corresponds to the non-circular guide wire in cross-sectional shape, the driving handle having a longitudinal axis; and rotating the drive handle about the longitudinal axis such as to thereby rotate the guide wire coupled with the driving handle and the bone screw coupled with the guide wire.

In accordance with a particular aspect of the present invention, there is provided a system for injecting fluid into a bone comprising: a fluid injector having a storage reservoir for the fluid in fluid communication with an injector tip from which the fluid is ejected when the fluid injector is actuated; and a bone screw having a screw body with at least one external thread on an outer surface thereof and a central bore extending at least partially into the screw body from an opening at an inlet end, the opening to the central bore being configured to matingly receive therein the injector tip of the fluid injector such that the fluid from the fluid injector is fed into the central bore when the fluid injector and bone screw are connected in fluid flow communication, and wherein one or more fluid ejection passages extend through the screw body from the central bore to the outer surface of the screw body, to the fluid ejection passages providing fluid flow in an outward direction from within the central bore to the bone surrounding the bone screw, when said bone screw is inserted into said bone and fluid is injected into the bone screw by the fluid injector.

There is also provided, in accordance with another particular aspect of the present invention, a method of injecting a fluid into a bone, comprising: providing a bone screw having a screw body with at least one external thread on an outer surface thereof and an at least partially cannulated central passage therein, the central passage having an opening at an inlet end thereof, the screw body having a number of fluid flow passages extending through a radial wall of the screw body such as to provide fluid flow communication between the central passage and the outer surface of the screw body; connecting a fluid injector with the bone screw by mating an injector tip of the fluid injector with said opening to the central passage at the inlet end of the bone screw, the injector having a reservoir for the fluid that is in fluid communication with the injector tip; and injecting the fluid into the bone by actuating the fluid injector to force said fluid within the reservoir to the injector tip and into the central passage of the bone screw, the fluid thereby flowing through the fluid flow passages in the radial wall of the screw body to the outer surface of the screw body, and therefore into the bone surrounding the bone screw.

There is also provided, in accordance with another particular aspect of the present invention, a system for inserting a bone screw into a tissue site comprising: a guide wire having a longitudinal axis and defining a non-circular cross-sectional shape in a plane substantially perpendicular to the longitudinal axis; and the bone screw including a screw body with at least one external thread thereon and having a central cannula extending longitudinally therethrough and configured for matingly receiving the guide wire therein, the cannula having a non-circular cross-section configured to rotationally couple the guide wire and the bone screw together such that relative axial rotation between the guide wire and the screw is prevented.

There is also provided, in accordance with another particular aspect of the present invention, a method of inserting a bone screw into a tissue site, comprising: positioning a proximal end of a guide wire into the tissue site at a desired location for insertion of the bone screw, the guide wire having a non-circular transverse cross-sectional shape and defining a longitudinal axis; providing a cannulated bone screw having a non-circular cannula, and inserting the bone screw onto a distal end of the guide wire and sliding the bone screw longitudinally along the guide wire to the desired location, and ensuring that the bone screw and the guide wire are rotationally coupled such that relative rotation therebetween about the longitudinal axis is prevented; engaging a drive handle to the guide wire such as to rotationally couple the drive handle and the guide wire; and rotating the drive handle about the longitudinal axis such as to thereby rotate the guide wire and thereby rotate the bone screw which is rotationally coupled with the guide wire.

There is also provided, in accordance with another particular aspect of the present invention, a bone screw comprising a screw body and having at least one external thread on an outer surface thereof, a cannulated passage extending through the screw body, and an inlet end of the screw body having an opening therein which opens into the cannulated passage and is configured to matingly receive therein an injector tip of a fluid injector for injecting a fluid into the cannulated passage of the bone screw, said cannulated passage having a non-circular cross-section configured to rotationally couple a guide wire having a non-circular cross-sectional shape such that relative axial rotation between the guide wire and the screw is prevented, the screw body having an annular portion which surrounds the cannulated passage therein and has a radial wall thickness, and a number of fluid flow passages extending through the radial wall and providing fluid flow communication between the cannulated passage and the outer surface of the screw body such as to direct fluid from within the cannulated passage in an outward direction through the screw body and into a tissue site surrounding the bone screw when the bone screw is inserted therein and the fluid is injected into the cannulated passage of the bone screw by the fluid injector.

There is also provided, in accordance with another particular aspect of the present invention, a kit for bone screw insertion within a tissue site comprising: a guide wire having a longitudinal axis and defining a non-circular cross-sectional shape; a bone screw having a screw body, at least one external thread on an outer surface thereof, and a cannulated passage extending longitudinally through the screw body, said cannulated passage having a non-circular cross-section configured to rotationally interconnect with the guide wire such that relative axial rotation between the guide wire and the bone screw is prevented, the screw body having an annular portion which surrounds the cannulated passage therein and has a radial wall thickness, the annular portion of the screw body having a number of fluid flow passages extending through the radial wall thickness to provide fluid flow communication between the cannulated passage and the outer surface of the screw body such as to direct a fluid within the cannulated passage through the screw body in an outward direction into the tissue site surrounding bone screw when the screw is inserted therein; and a drive element which rotationally engages the guide wire for rotation of the guide wire about the longitudinal axis thereof, and thereby also rotating the bone screw when the bone screw and the guide wire are interconnected.

The kit for bone screw insertion may also comprise a fluid injector for injecting the bone growth promoting fluid into the cannulated passage of the bone screw, the bone screw having an inlet end which includes an opening in communication with the cannulated passage and matingly receiving therein an injector tip of the fluid injector, the fluid injector including a storage reservoir for the bone growth promoting fluid in communication with the injector tip from which the bone growth promoting fluid is ejected when the fluid injector is actuated.

### DESCRIPTION OF THE DRAWINGS

Reference is now made to the accompanying drawings, showing by way of illustration a preferred embodiment thereof, and in which

FIG. 1 is a perspective view of an injectable bone screw in accordance with an embodiment of the present description;

FIG. 2A is a longitudinal cross-sectional view of the injectable bone screw of FIG. 1;

FIG. 2B is an end view of the injectable bone screw of FIG. 1;

FIG. 3A is a side view of an alternate bone screw of FIG. 1;

FIG. 3B is a cross-sectional view of the bone screw of FIG. 3A taken through line 3B-3B thereof;

FIG. 4 is a cross-sectional view of a bone screw in accordance with an alternate embodiment;

FIG. 5A is a perspective view of a screw insertion system comprising the injectable bone screw of FIG. 1, shown with a guide wire inserted into a drive handle for driving rotation of the guide wire by the handle;

FIG. 5B is a partial perspective view of the screw insertion system of FIG. 5a, shown with the bone screw slid onto the guide wire which is inserted into the drive handle;

FIG. 6A is a schematic perspective view of the guide wire of the system of FIG. 5A, the guide wire having a hexagonal cross-sectional shape in accordance with one particular embodiment;

FIG. 6B is a schematically cross-sectional view of a plurality of different cross-sectional shapes of the guide wire of FIG. 6A, in accordance with alternate embodiments;

FIG. 7A is a schematic plan view of a syringe used to inject a bone repair promoting material into a fractured scaphoid of a patient's wrist through the bone screw of FIG. 1; and

FIG. 7B is an enlarged view of the syringe used to inject the bone repair promoting material into the fractured scaphoid through the bone screw, taken from region 7B-7B of FIG. 7A.

### DETAILED DESCRIPTION

The present disclosure relates generally to a bone screw and its associated system and method for insertion thereof and for injecting a fluid into a bone site using the bone screw, as described herein. The viscous fluid may be, for example, a bone repair promoting material. However, as noted below other fluids and/or viscous media may be injected into the bone site. In at least one possible embodiment, the screw is permeable to the fluid such that this fluid may be injected into the bone screw, which in at least one embodiment is at least partially porous, and thereby ejected outward from the screw and directly into the surrounding tissue or environment surrounding the screw. Such tissue can include, for example, hard tissue such as bone, softer tissue such as ligaments and tendons, or a combination of the two. In the case when the screw is inserted into a hard tissue such as bone, this may be done to help heal a fracture site in the bone for example. Accordingly, although the term “bone screw” may be used herein given the preferred embodiment wherein the screw is inserted into bone, it is to be understood that the presently described screws can also be inserted into and used in a non-bone tissue environment.

The presently described fluid injection system, which in this embodiment includes the permeable and/or porous bone screw, may be used to inject a fluid into the surrounding tissue, be it bone or otherwise. The term “fluid” as used herein is understood to include any suitable fluid or viscous media/material which may be injected into a bone or tissue site. Such fluids may include but are not limited to: a bone repair promoting material, such as a bone cement used for fracture fixation for example; or a bone-treating material, such as an antibiotic and/or a chemotherapy agent for example, that may be used for the healing or treatment of the bone into which they are injected. The fluids which are injected into the bone/tissue using the presently described systems and methods may also comprise other fluids having active ingredients used for treating any number of musculoskeletal pathologies, including but not limited to, fractures, infections, cancer, metabolic diseases, generative disorders, etc. It is also to be understood that these fluids and viscous media include any fluid-like material (ex: pastes, suspensions, etc.) which can be forced through the injector and out of the porous bone screw, be it paste-like and thus highly viscous or slightly less viscous and thus more free-flowing. Such fluids can also include, but are not limited to, biological materials such as bone graft, blood, bone marrow or stem cells, natural bone grafts or paste, artificial bone graft substitutes such as those made with natural materials (demineralised bone matrix and coral for example) or synthetic materials (calcium phosphate, hydroxyapatite, bioglass for example), bone growth factors such as bone morphogenetic proteins (BMPs), biological tissues, pharmaceutical agents, therapeutic agents, bone cement, markers, additives such as those which ease injection of the media through the porous material or fluid ejection passages of the bone screw, and/or any mixtures or combinations thereof.

Other examples of the fluids which can be injected using the present system include: biological materials such as blood, marrow and stem cells, which have the ability to promote healing and could be used to promote tissue formation; bone cements (such as PMMA, for example), both permanent or resorbable, which can help provide initial fixation and stability to thereby improve healing; and therapeutics or pharmaceutical agents, which can be used to improve the healing process. The injectable bone screws as defined herein are therefore permeable to all such viscous media, such as to permit the injection of the selected viscous media via the bone screw and into the surrounding tissue.

As will be seen in further detail below, the injectable bone screws described herein are permeable and also include at least one cannulated passage therein (and for example extending longitudinally therethrough, either partially or fully) such that any of the above-noted bone-treating fluids injected into the bone screw can be directed through the internal cannulated passage and outward therefrom and thus injected directly into the bone or soft tissue surrounding the bone screw from within. In at least one possible embodiment, the bone screw is porous and thereby provides a plurality of such fluid flow cannulated passages therein.

As will be seen, the injectable screw 10 particularly comprises a central passage 16, which is at least partially cannulated (i.e. extends at least partially into the screw body. In one embodiment, the central passage 16 is fully cannulated such as to receive a guide wire 402 having a non-circular cross-section, in which case the fully cannulated passage 16 also has a non-circular cross-section which is configured to receive therein, and may or may not correspond to, the guide wire and which therefore rotationally interconnects (i.e. rotationally couples) the guide wire and the screw together such that relative axial rotation between the guide wire 402 and the screw 10 is prevented when the guide wire 402 is inserted through the cannulated passage 16.

The presently described injectable bone screws accordingly permit the reduction of stresses during the insertion. When a screw is normally driven through a solid, significant torsional stresses can be generated. These stresses can cause the failure of the screw or damage the connection of the screw. In both cases, this may cause complications during the surgery. The maximum stress in the screw during its insertion is directly proportional to the length of the screw, the diameter of the cannula and screw as well as the moment of torsion in the screw. The length of the screw and the diameter of the screw and its cannula are mostly fixed by anatomical considerations and clinical conditions. The torsion moment can, on the other hand be modified by the way the screw is inserted. Thus, one way to minimize the stresses and deformation of the screw during the insertion is to distribute the stress over a larger surface and all along the length of the screw.

It has been found that the distribution of stress over a larger surface and along the length of the screw can be achieved if the internal section of the injectable screw is not circular. Accordingly, as will be seen in further detail below, the injectable bone screw of the present disclosure comprises a central cannula that defines a non-circular cross-sectional shape. The guide wire which fits within this injectable bone screw cannula has a corresponding cross-sectional shape, and is therefore also not circular.

The presently described injectable bone screws therefore define a fluid flow cannulated passage which is not circular and the driver used to insert these screws, which is the guide-wire itself, can be inserted along the complete length of the cannulated passage and therefore of the injectable screw (or at least an extended portion of the screw). This permits the torsional moment to be very significantly reduced, and in fact may be reduced to near zero. In this case, the stresses are mostly flexional and will depend on the thickness of the wall of the injectable screw (i.e. difference between the external and internal radius of the screw).

With this configuration, the stress is distributed on a larger surface of the injectable screw and the lever for the generation of the stresses is much shorter, namely the thickness of the wall of the screw instead of the length of the screw. Consequently, this configuration allows for reduction of the stress and deformation in the injectable screw, and therefore reduction of the risks of failure of the screw during insertion.

The injectable bone screws described herein may be principally intended for use in orthopaedic applications, such as for fracture fixation in, for example only, a scaphoid or vertebra. However, besides fracture repairs, the bone screws as described herein and the described insertion methods and injection methods may also be used for other medical uses, including for example for bone reconstruction (i.e. osteotomy, augmentation or solidification for example), fusion (vertebra, small bones of the hand, wrist and feet for example), to fix implants (arthroplasty, hand and foot surgeries, spine, trauma for example), fix soft tissues such as ligaments, tendon or cartilage to bone, etc. As described, the bone screws may be used as a fluid injection system only (i.e. need not be used for any facture fixation), such as to inject various fluids/materials directly into a bone site. For example, the bone and related injection system described herein may be used to inject chemotherapy agents into cancerous bone, to inject antibiotics into an infected bone, etc. Accordingly, the presently described systems and methods can be used for treating any number of musculoskeletal pathologies, including fractures, infections, cancer, metabolic diseases, generative disorders, in addition to the fixation of fractures and the reduction of fracture gaps as described further herein. The present injectable bone screws may therefore be used in a wide variety of applications for treating musculoskeletal disorders, including for example screws used to repair diseased bones, such as for femoral head necrosis, augmentation of vertebra, and osteotomy.

The terms “bone screw”, “orthopaedic screw” and/or “biomedical screw” as used herein are intended to encompass screws which may be used for any or all of such possible uses. Further, although the insertion and injection systems defined herein will generally be described with respect to a bone screw inserted into a bone site, it is to be understood that such screws can also be used to fix soft tissues to bones, such as tendon, ligaments and cartilages, and therefore may be inserted within soft tissue, or a combination of soft and hard tissue, as well solely in bone. It is also understood that said bone screws can be inserted to strengthen bone weakened by a cancerous lesion or damaged by infection, metabolic disorder or degenerative diseases. Therefore, while the injectable screws described herein may be bone screws used in joining together fractured fragments of a broken bone, many other applications of the present fluid injecting screws exist, as per the examples provided above.

Additionally, although the depicted injectable bone screws are compression screws, it is to be understood that the present injectable bone screws might also be a non-compression screw, for example a screw used to fix implant or used in bone reconstruction or used for fixation of connective tissues or cartilage. For example, the presently described injectable bone screws may also be employed to fasten external fixators in place to a bone, or to fasten other medical implants in place, such as rods used to stabilize the vertebral column for example.

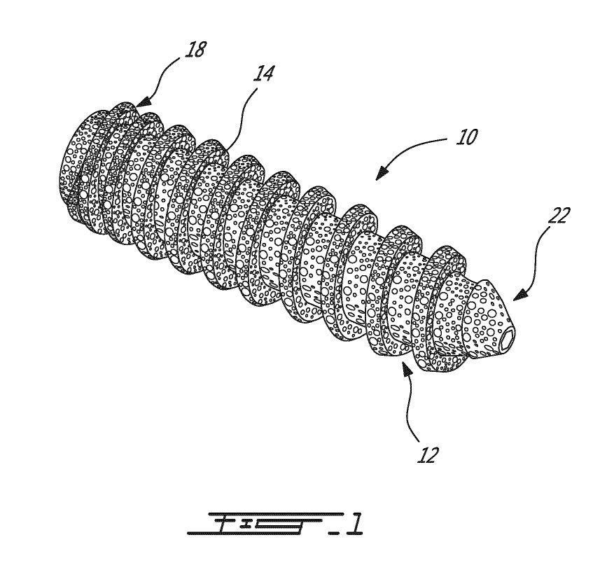

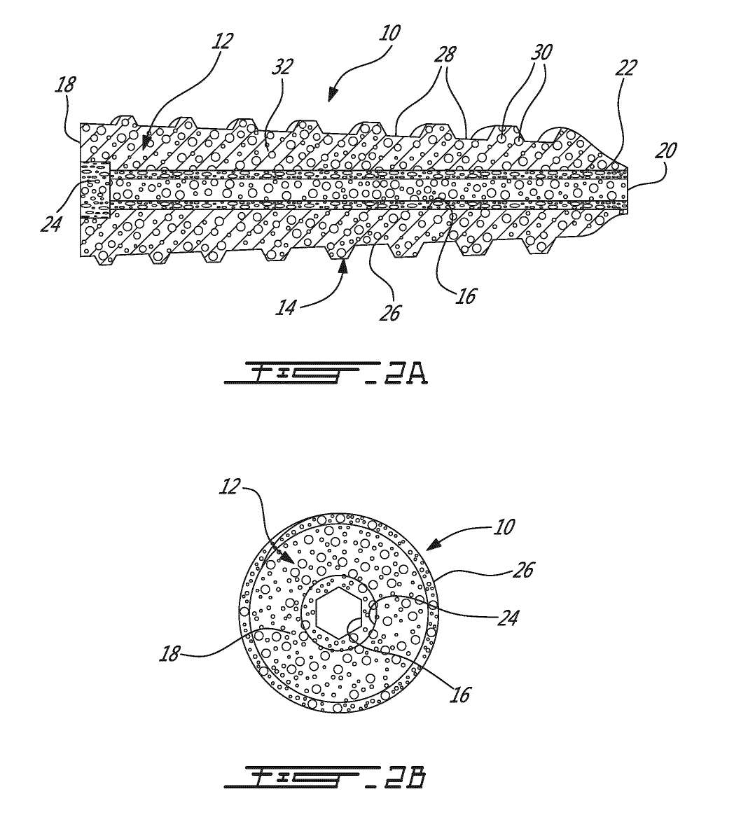

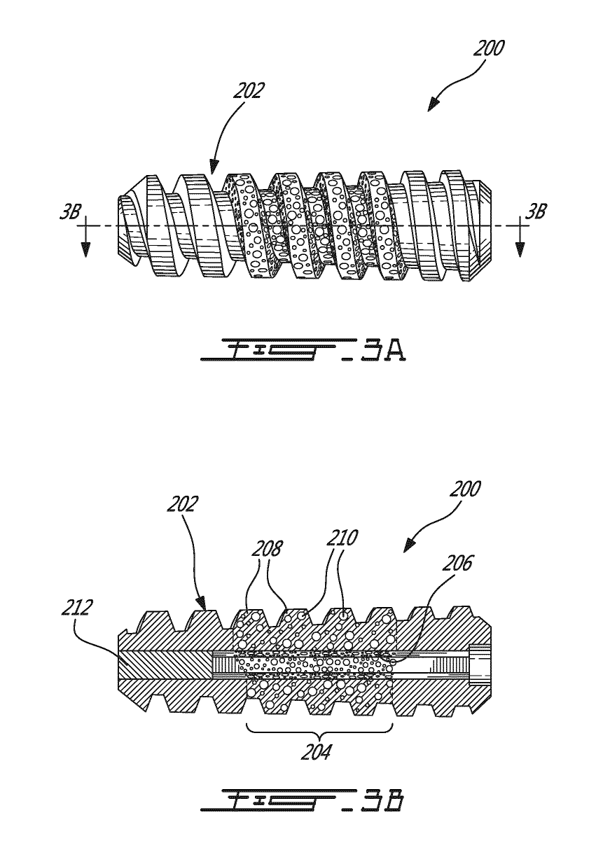

Turning now to FIG. 1, the injectable screw 10 in accordance with one embodiment of the present disclosure is depicted. In this embodiment, the injectable bone screw 10 includes a screw body 12 which may be an integrally formed, one-piece body and which has at least one external thread 14 on outer peripheral surface thereof. As seen in FIG. 2A, a central cannulated passage 16 having a non-circular cross-section, and more precisely an hexagonal shape in at least this particular embodiment, longitudinally extends through the full length of the injectable bone screw 10 between a head end 18 of the screw body 12 and the tip opening 20 of the cannulated passage 16. As best seen in FIG. 2B, the cannulated passage 16 of the injectable screw 10 has a non-circular cross-section, and which in at least one embodiment also defines a hexagonal shape. A guide wire as further detailed hereinbelow having a non-circular cross-section is inserted in the cannulated passage 16, rotationally interconnecting the guide wire and the screw together, such that relative axial rotation between the guide wire and the screw is prevented when inserting the injectable bone screw 10 in the bone or in position during surgery for example.

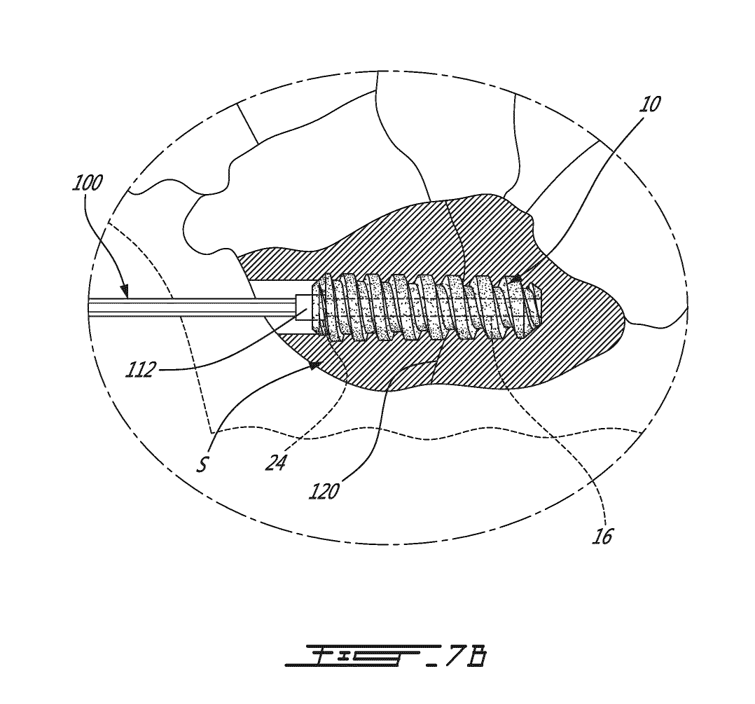

In an embodiment, the injectable screw 10 comprises an adapter opening 24 which may have a diameter which is greater than that of the cannulated passage 16 and which is sized and configured to receive an adapter that is used to fluidicly interconnect an injector or syringe 100 and the cannulated passage 16 of the injectable bone screw 10, as described in further detail below. The cannulated passage 16 in the screw body 12 defines an inlet end through which fluid from the syringe 100 is received when the syringe 100 and the bone screw 14 are connected in fluid flow communication (FIGS. 7A and 7B).

In the embodiment of FIGS. 2A-2B, the screw body 12 of the injectable bone screw 10 is one-piece or monolithic (i.e. integrally formed from a single piece of material), however the properties of this monolithic screw body 12 may not necessarily be uniform throughout. Nonetheless, the screw body 12 is necessarily at least partially permeable, and defines one or more fluid ejection passages 28 through the screw body 12 which extends from the cannulated passage 16 to the outer surface 26 of the screw body 12. In the case of the injectable bone screw 10, a plurality of these fluid ejection passages 28 are provided and defined by a plurality of pores 30 which are interconnected and disposed throughout the entirety of the screw body 12. As such, the screw body 12 of the injectable bone screw 10 is in fact composed of a fully porous material, such that the injectable bone screw 10 is porous throughout. These pores 30 are interconnected to define a foam-like matrix through the full radial wall thickness of the annular portion 34 surrounding the cannulated passage 16 of the screw body 12. The injectable body screw 10 may be made, for example, from a substantially rigid foam material, which provides the plurality of pores 30 such that they are interconnected together to form a plurality of fluid ejection passages 28 extending through the screw body 12 of the injectable bone screw 10, thereby permitting the viscous media, injected by the syringe 100 into the cannulated passage 16 of the injectable bone screw 10, to flow through these passages 28 in the screw body 12 and out of the screw 10 into the surrounding environment, i.e. the tissue in which the injectable bone screw 10 is located.

The screws as defined herein may be configured and/or formed in a manner similar to the bone screws as defined in International Patent Application No. PCT/CA2010/001645 filed Oct. 13, 2010, the entire content of which is incorporated herein by reference.

Therefore, the injectable bone screw 10 of the present disclosure is in fact used as an injector to inject the viscous media or fluid into the tissue surrounding the screw or into nearby gaps which may form between fragments of a fractured bone in which the screw is inserted, for example.

In one possible embodiment, the interconnected pores 30 in the injectable bone screw 10 also allow some bone or other tissue ingrowth into the screw body, thereby potentially minimizing the space occupied by the screw within the bone or other tissue being fixed within the screw. However, it is to be understood that this tissue or bone ingrowth will only occur after the above described viscous media is injected into the surrounding tissue via the pores 30, and therefore the fluid ejection passages 28, formed in the injectable bone screw 10. In another embodiment, however, the pores 30 may be sized such that they are sufficiently small to in fact prevent any bone or tissue ingrowth, while nevertheless allowing for the injection of the viscous media through the screw following its insertion. This embodiment will be described in further detail below.

The injectable bone screw 10 depicted in FIGS. 2A-2b is a compression bone screw which has a varying pitch of the thread 14 formed on its outer periphery, such as to compress together two portions of fractured bone into which the bone screw is installed. Although the depicted bone screw 10 happens to be headless, it could nonetheless be used as an injector to inject the viscous media, such as a bone healing promoting material, or antibiotic, or anticancer agent, or any other drug or compound to treat a condition affecting bone, into a surrounding tissue site while nevertheless having a head, but this will of course be dependent on the particular application desired for the screw.

As noted above, the injectable bone screw 10 may be formed for example at least partially by rigid foam which defines the matrix forming the plurality of interconnected pores 30 therein. These pores are interconnected to form the plurality of fluid ejection passages 28 through the entirety of the screw body 12, such as to permit the flow of a viscous media outwardly from within the cannulated passage 16, through the annular wall portion 32 of the screw body 12, and out into the surrounding environmental tissue. While the bone screw 10 is depicted as being entirely formed by the above-mentioned rigid foam such that it is porous throughout, it is to be understood that only a portion of the screw body may in fact have the interconnected pores 30 and therefore the fluid ejection passages 28 therein and therefore that only a portion of the screw body may be formed by such a rigid foam. The rigid foam may be composed of a porous metal, ceramic or polymer material, which define the pores formed therein that are interconnected to define the fluid ejection passages that permits the flow of the viscous media through the body of the screw and outward to its surrounding environment.

The pores 30 of the porous screw body 12 portions described herein interconnect to form a plurality of interconnected voids, each in communication with the next adjacent void, and which extend substantially uniformly in all directions of the given porous section of the screw 10, and at least in the radial direction from the outer surface of the screw 10 to the inner cannulated passage 16 thereof. In at least one particular embodiment, the pores 30 are substantially uniformly sized and substantially uniformly spaced apart, as much as is reasonably feasible based on the production process used to form the bone screw 10. However, it is to be understood that the pores 30 of the rigid foam material which makes up the injectable bone screws 10 described herein do not necessarily need to be of equal size or equally, or homogenously, spaced apart. The rigid foam, which may but is not necessarily composed of metallic foam, may be comprised of a porous sintered metal made from metal powders using powder metallurgy techniques for example. The metallic foam may contain titanium, magnesium, iron, tantalum or an alloy of one or more thereof such as stainless steel, Ti6Al4V, TiNi, or a ceramic. This metallic foam material forms a metal matrix or network that defines inter-connected pores 30 throughout. This interconnected porosity allows fluid flow from one side of the screw body 12 to the other, and therefore allows for full bone in growth (in fact permits bone through-growth). This is in contrast, for example, with isolated surface pores (ex: machined or otherwise formed in a solid metallic part), which do not have connectivity between each other and with both surfaces of the component. In one embodiment this porous material is metallic foam, made for example of a titanium alloy. Although other ranges are of course possible, the pores 28 have a size (ex: diameter), in at least one particular embodiment, of about 30 to about 500 microns (i.e. μm), but preferably between 50 to 400 μm, to achieve desired levels of bone in growth and mechanical strength, and the porous material has a porosity ranging from 30% to 80%, but preferably between 40 to 70% to obtain a desired level of mechanical strength. In another possible embodiment, pores and/or the fluid flow passages have a cross-sectional size (i.e. size in at least one direction, such as width for example, or alternately in diameter for circular openings) of between 5 and 2000 microns, and more preferably of between 10 and 1000 microns, more preferably between 30 and 500 microns, and more preferably still between 50 and 400 microns.

It goes without saying that the material selected for the bone screw 10 must be biocompatible, and may, but is not necessarily, non-ferromagnetic and thus allows magnetic resonance imaging (MRI) of the bone.

The bone screw 10 is therefore formed such that it is at least partially permeable to the viscous material which is desired to be injected into the surrounding bone or tissue into which the bone screw 10 is inserted. Accordingly, the size of the pores 30 and/or the fluid ejection passages 28 as well as the consistency and viscosity of the material to be injected, are selected such that the material is able to flow through the screw body 12 and outward into the surrounding tissue. This may be used for example to inject the viscous media into fractures present in the bone in which the bone screw 10 is located, whereby the bone screw 10 is inserted or disposed between the bone or other tissues which are required to be joined to the bone. The injection of the viscous media into the fractured site is therefore performed to at least partially fill the fracture gap, thereby improving the stability of the bones on either side of the fracture and/or to promote fusion between the bone segments or the bone and other tissues. Alternately, the injectable bone screw 10 may be used between adjacent bones in order to help fuse them together, whereby the injection of the viscous media in the gaps formed between the two bones may help improve their stability or bond them together. Similarly, the bone screw 10, associated with an injector or a syringe 100, can be used to inject a viscous media into softer connective tissue which may need to be connected to a bone. Accordingly, the injection of the viscous media may be to at least partially fill a gap between the screw 10 and the surrounding tissues, to help promote healing and fixation.

Although a number of possible uses exist for injecting a viscous media into the surrounding bone or tissue using the present injectable screw 10, in one possible embodiment the present injectable screw 10 may be used for the injection of a viscous media containing tracers for imaging purposes. For example, the present screw 10 may be used to inject a media containing barium oxide into the surrounding tissue in order to improve imaging of the tissue in question using X-rays. Other tracers can of course also be used in stead of barium oxide, for imaging X-rays or other imaging techniques.

The porous nature of the screw body 12 of the bone screw 10 is such that it must allow the flow and therefore injection of viscous media through the fluid ejection passages 28 formed by the interconnected pores 08, such as to permit fluid flow between the cannulated passage 16 and the outer surface 26 of the screw 10. However, in one particular embodiment, ingrowth of the surrounding tissues into the pores 30 of the screw body 12 may need to be prevented, for example in the eventuality that these screws need to be removed. In such a case, the material used to form the screw body 12 is selected such that the pores 30 defined therein are sufficiently large to permit a flow of the selected viscous media through the screw body 12 for injection of this media into the surrounding tissue, while the pores 30 being nonetheless sufficiently small to prevent the surrounding tissue from growing into the pores 30 of the screw body 12 following its insertion. For example, in one possible embodiment, the pores 30 may have a size less than 15 micrometers, which is sufficiently small to prevent bone ingrowth while still permitting fluid flow through the interconnected pores 30 and thus ejection of the viscous media from the passages formed in the screw 10 by these pores 30.

As noted above, following insertion of the bone screw 10, the viscous media is injected by the syringe 100 into the cannulated passage 16 of the screw, in order to impregnate the pores 30 of the screw 10 from the inner cannula outward. The bone screw 10 is therefore permeable to the viscous media, which is thereby ejected out of the bone screw 10 in situ within the bone or surrounding tissue. This injection of the viscous media in the screw 10 may be done to fill, at least partially, the gap of a fracture to improve the stability of the bones on each side of the fracture or to promote the fusion between the bone segments. The injection of the viscous media in the screw 10 can also be done to fill, at least partially, the gap between adjacent bones to improve their stability or to fuse them together. The injection of the media in the gap could also be done to fill, at least partially, the gap between the screw 10 and tissues in contact with the screw 10, to promote healing and fixation, such tissues being tendons, ligaments or cartilages. The viscous media can also be done to diffuse in the surrounding tissues to heal tissues surrounding the screw 10. In all cases, though, the viscosity of the media must be such that it can be injected through the porosity of the screw 10. If charges are present in the media (demineralised bone matrix, calcium phosphate, tricalcium phosphate, hydroxyapatite, bioglass particles or tracer particles, for example), their particles size must be such that they can, at least partially, flow through the pores 30 and/or the cannulated passages of the injectable bone screw 10.

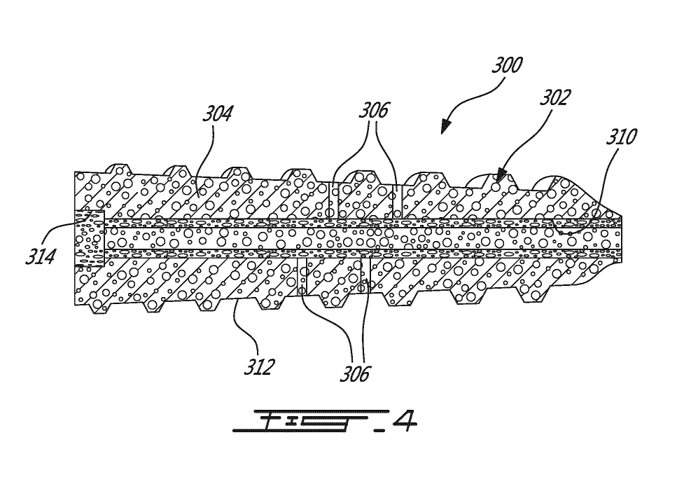

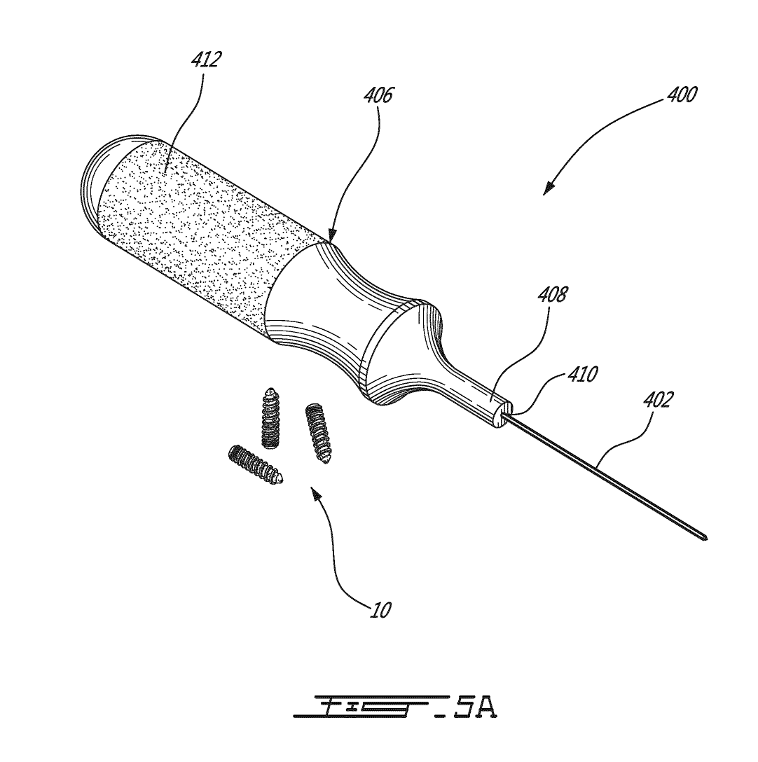

Referring now to FIGS. 3A-3B, an injectable bone screw 200 in accordance with an alternate embodiment is depicted. The bone screw 200 is substantially similar to the bone screw 10 described above, however as is best seen in FIG. 3b, the bone screw 200 defines a screw body 202 that is porous only in a portion or region thereof, for example a central portion 204 of the screw. Accordingly, the screw body 202 remains permeable to the viscous material which is injected into the cannulated passage 206, however only within this central region 204 in which a plurality of pores 208 are provided and in which they interconnect to form the fluid ejection passages 210 for the viscous fluid to be ejected through the bone screw 200. Although only the central portion 204 of the bone screw 200 is porous in the depicted embodiment of FIGS. 3A-3B, it is also possible that other regions of the bone screw 200 may alternately be made porous rather than the central portion. For example, it may be desirable to provide a bone screw in which the opposing ends thereof permit the ejection of the viscous material therefrom while the central portion of the bone screw remains substantially solid and free of pores and/or fluid ejection passages.

Accordingly, the configuration of the injectable bone screw can be tailored in order to suit the particular environment or application in which it is intended to be used, thereby permitting the controlled and localised injection of the selected viscous media into the surrounding tissue only in places where this is desired. The bone screw 200 includes a passage 206 which extends at least partially along the length of the screw body 202, however the passage 206 does not extend the full length of the screw 200 and therefore is closed in one end thereof (i.e. the screw is only partially cannulated). This may be accomplished either by forming the bone screw 200 such that the bore only extends partially into the screw body at manufacturing, or alternately the annulated passage 206 may be initially formed such that it extends the full length of the screw body 202 but subsequently sealed by a bore plug 212 which is subsequently inserted into the annulated passage 206 if it is desired to enclose one end of the annulated passage 206 for example. This bore plug 212 may be inserted into the annulated passage 206 of the bone screw 200 either before or after insertion of the bone screw 200 into the surrounding tissue.

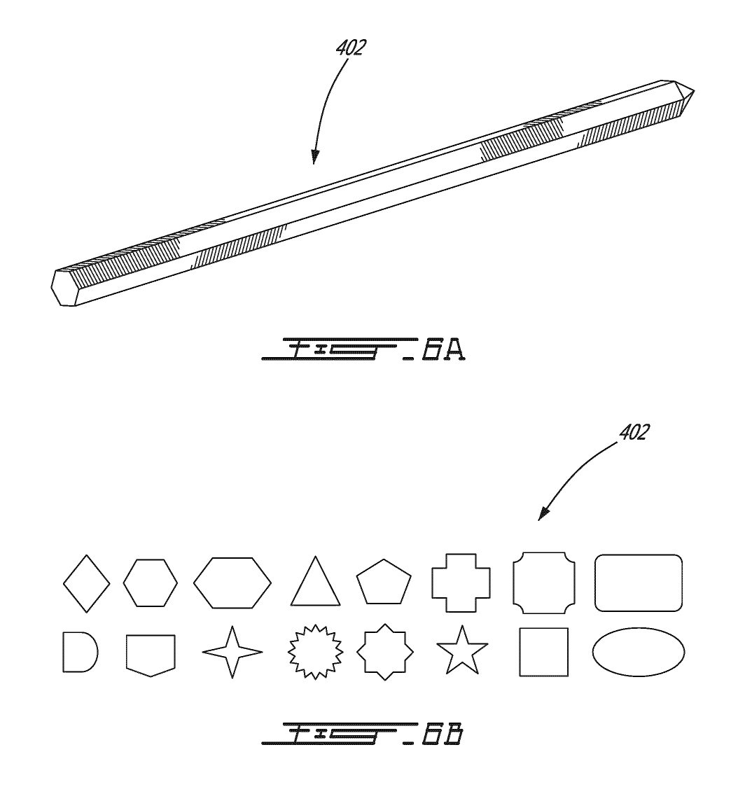

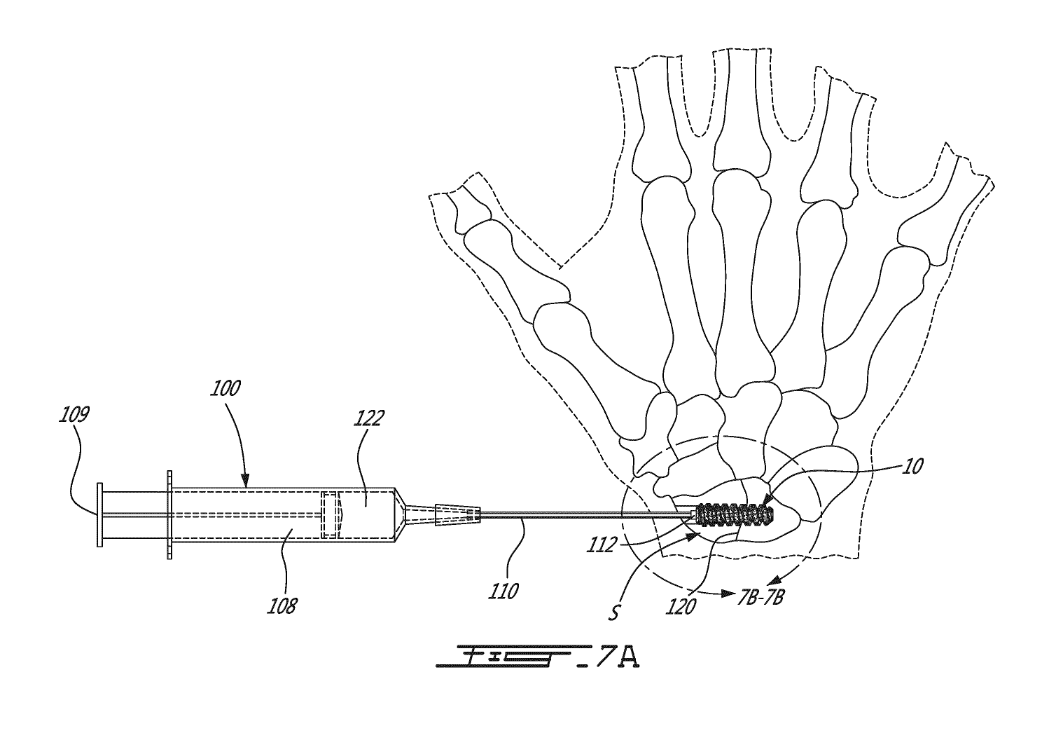

In yet another embodiment of a bone screw 300 as described herein, FIG. 6 depicts a bone screw 300 which remains at least partially permeable to the viscous media to be injected into the surrounding tissue, however the bone screw 300 is formed having a screw body 302 which is substantially solid but defines therein a smaller number of fluid ejection passages 306 which extend through the annular body portion 304 of the screw body 302 between an annulated passage 310 and an outer surface 312 on the periphery of the bone screw 300. Much as per the injectable bone screws 10 and 200 described above, the annulated passage 310 of the bone screw 300 permit fluid flow communication between the annulated passage 310 and the surrounding tissue into which the bone screw 300 is inserted, such that when the viscous media is injected into the annulated passage 310 of the bone screw 300 by connecting a syringe 100 to the adapter opening 314 in the bone screw 300, this viscous media is forced into the annulated passage 310 and subsequently out through the fluid ejection passages 306 in the screw body 302 and therefore into the surrounding tissue. However, in this embodiment the screw body 302 is not formed by a porous structure, but may simply comprise a simple solid screw body having individually formed fluid ejection passages 306 therein. These passages may be formed either concurrently with the forming of the screw body, such as by a machining or another shaping process, or alternately the passages 306 may be separately formed such as by using a machining or etching process during the manufacture of the bone screw 300.

While the above-described bone screw and the associate method and system used for injecting the bone-treating fluid using same may be inserted in a number of ways, one particular manner which may be used to insert the bone screw into a bone will now be described. While the installation method described below relates to the use of a guide wire to install the fully cannulated embodiment of the bone screw with the bone site, it is understood that other installation and insertion techniques may also be used. For example, in the case of a partially cannulated screw (i.e. wherein the central bore or cannula does not extend fully the through the length of the screw), the above-described guide wire would not be used. Alternate installation techniques which may also be used to install such non-fully cannulated bone screws include the use of a screwdriver, having the same shape as the cannula opening for example, or with a convention screwdriver which engaged a head of the screw.

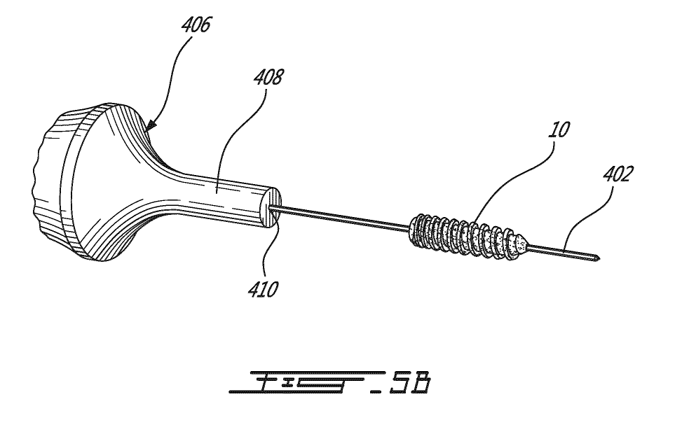

Referring now to FIGS. 5A and 5B, a screw insertion system/kit 400 is depicted which includes generally a guide wire 402 and at least one injectable bone screw 10 as described herein. An associated drive element includes a drive handle 406 used for the insertion of the screws 10 may also included, which is removably engaged with the guide wire such as to rotate the guide wire about its longitudinal axis. As noted above, the guide wire 402 has a transverse cross-sectional shape that is non-circular. In the present embodiment, the guide wire 402 defines a hexagonal shape, as described in further detail below with respect to FIG. 6A. The cannulated passage 16 of the screw 10 also has the same non-circular cross-section, and there which in at least this particular embodiment also defines a hexagonal shape as better seen in FIGS. 1 and 2B.

Therefore, as better seen in FIG. 5B, the hexagonal guide wire 402 can be inserted into the hexagonal cannulated passage 16 of the screw 10, such that the guide wire 402 engages the screw 10 for mutual and corresponding rotation. In other words, the non-circular shapes of both the cannulated passage 16 of the screw 10 and the mating guide wire 402 are such that rotation of the guide wire 402 will cause the same corresponding rotation of the screw 10. The size of the cross-sectional shapes of the screw cannulated passage 16 and the guide wire 402 are configured so that the guide wire cannot rotate within the cannulated passage 16 of the screw 10. For example, the inner size and shape of the cannulated passage 16 engages with the outer, non-circular, configuration of the guide wire 402, such that rotation of the guide wire 402 will cause the screw 10 to rotate accordingly.

The handle or drive element 406 includes a drive end 408 having a cannula opening 410 therein which is shaped and configured to matingly receive therein the guide wire 402, as best seen in FIG. 5a, and is therefore also non-circular in shape. The cannula opening 410 may be centrally disposed within the drive element 406 and may, although does not necessarily, extend the full length of the handle body. As will be seen, the drive element 406 acts as a screw driver, the screw driver “bit” of which being the guide wire 402. Accordingly, the drive element 406 includes a grip portion 412 which permits a user to turn the handle about its longitudinally axis, which may be coaxial with the cannula 410 therein, such as to drive the guide wire 402 received therein.

As such, the guide wire 402 can be inserted into both the cannulated passage 16 of the screw 10 and the cannula opening 410 of the drive element 406. In the depicted embodiment, the cross-sectional shapes of the guide wire 402, the screw cannulated passage 16 and the handle cannula 410 are all identical. However, as noted below, these cross-sectional shapes may differ provided that the respective cross-sectional shapes of the guide wire 402 and the cannulated passage 16 are cooperative such that the guide wire 402 fits within the cannulated passage 16 without permitting relative rotation of the guide wire 402 and the screw 10 and/or drive element 406.

Accordingly, as the cross-sections of the guide wire 402 and the cannulated passage 16 of the screw 14 and the cannula 410 are all not circular, the guide wire 402 is not able to rotate freely in either the screw 10 or drive element 406. Thus when the guide wire 402 is inserted into the cannulated passage 16 of the screw 10, rotating the guide wire 402, which can be achieved by rotating the handle or drive element 406 when the guide wire 402 is also inserted therein, will cause the screw to be driven into the tissue. The injectable bone screw 10 can thus be inserted into tissues, such as bone, soft tissue, etc, by rotating the drive element 406, the guide wire 402 which is disposed there between acting as a torsional force transmitting element (much like a removable screw driver bit, for example). The tip 408 of the drive element 406 can be used to apply a pressure on the screw 10 to ease its insertion.

The tip 408 of the drive element 406 and the head end 18 of the screw 10 can also be textured or shaped in such a way to provide additional engagement with the guide wire 402, to further reduce torsional stresses and deformation in the guide wire 402 during insertion of the screw into the tissue. Such features can include, for example, steps, hexagonal protrusions, crosses, stars or fins.

Although the screw insertion system/kit 400 depicted in FIGS. 5a and 5b includes a drive element 406 which takes the form of a handle which is a hand tool that is manually operated for manual insertion of the screw 10, the drive handle or drive element used can also include mechanical or electric systems which are able to grip and rotate the guide wire 402 for the insertion of the screw 10. While the use of a cannula 410 in the drive element or handle 406 is a simple solution for driving the guide wire 402, other methods can also be employed for securing the guide wire 402 in the handle 406. It is of course to be understood that the size of the guide wire and cannula in the handle can be adapted to the type and size of the screws.

The guide wire 402 is used as a guide along which the injectable bone screw 10 can slide such as to direct the insertion of the screw, much as per traditional guide wires. However, the present non-circular guide wire 402 is also used as a driver, to drive the screw 10 into bone and/or other tissue. The non-circular cross-sectional profile (see FIGS. 6A and 6B, for example) of the present guide wire 402 and the associated screw 10 having a non-circular cannulated passage 16 therein, provide a configuration which allows for stress reduction during the insertion of the injectable screw 10. While the system and method described herein may be used for all types of injectable screws as encompassed herein, it may be particularly useful for the insertion of screws with reduced mechanical properties such as those produced with polymers, ceramics and/or porous materials.

The use of the present screws 10 having a non-circular cannulated passage 16 with corresponding non-circular guide wires 402 helps to overcome significant technical challenges typically encountered when using known circular guide wires and cannulated screws. This is particularly true as the diameter of the screws used in biomedical applications is very small, and therefore to be able to insert simultaneously both the guide wire and the screw driver inside the whole length of the screw cannula, as is often done in known devices, to drive the screw becomes very difficult. However, the present system/kit 10 and the method of inserting screws using such tools, solves this problem.

Referring now again to FIG. 6A, the guide wire 402 has a hexagonal cross-sectional shape along its complete length. While in this embodiment the cross-sectional shape of the guide wire 402 is uniform through the complete length of the wire, the section can alternately vary along its length provided that the screw can be engaged and driven by the guide wire. The shape of the cross-section of the guide wire will however be the same as the section of the cannulated passage of the screw.

FIG. 6B depicts a number of other guide wire 402 cross-sectional shapes which can also be used, besides hexagonal sections. Although several examples of possible non-circular cross-sectional guide wire shapes are shown in FIG. 6B, it is to be understood that any type of non-circular section could be used as long as the guide wire can be engaged in the screw and cannot rotate freely in the cannula of the screw. Sections of the guide wire can for example be hexagonal, octagonal, pentagonal, triangular, square, cross shaped, star shaped, oval, any combination thereof, or any other cross-sectional shape which does not allow the rotation of the guide wire inside the cannula of the screw.

In the presently depicted embodiment, the cannulated passage 16 of the screw 10 has a cross-sectional shape which corresponds to that of the guide wire 402. However, the shape of the section of the guide wire can be different from the shape of the section of the screw, as long as the guide wire can be inserted in the screw and cannot freely rotate in the screw in such a way that the guide wire can engaged the rotation of the screw for its insertion in the tissues. For example, the guide wire may have an X-shaped section which can be inserted into a cannula having a square-shaped section of the same diagonal.

The guide wire 402 can be rigid for ease the insertion. Guide wires with some flexibility can also be used to get access to sites not directly in line of sight with the insertion location. The diameter of the guide wire may be between 0.5 mm and 5 mm depending on the size of the screw. The guide wire can also be hollow or perforated.

Once the injectable screw has been inserted into place within the bone using the insertion kit described hereinabove, a bone repair promoting fluid may then be injected into the bone site using the injection kit, system and/or method as will now be described.

Referring now to FIGS. 7A and 7B, the use of the above-described injectable screw 10 to inject the bone-treating fluid into a fracture site in the scaphoid in a patient's wrist, is shown. In one particular embodiment, the inlet end of the injectable bone screw 10 is configured to engage a fluid injector 100, connectable together in fluid flow communication. The fluid injector 100 may consist of a syringe having a cylindrical reservoir portion 108 and an elongated needle portion 110 having an injector tip 112 at a remote end thereof.

As best seen in FIGS. 2A-2B, the adapter opening 24 in the head portion 18 of the body 12 of the bone screw 10 is formed having a diameter which is greater than that of the cannulated passage 16, and is formed and configured in order to matingly receive the injection tip 112 of the fluid injector or syringe 100 therein. In one possible embodiment, the adapter opening 24 forms a tight fit with the injector tip 112, such that the two remain in coupled and mating contact during the entire injection process, whereby the fluid injector is actuated to force the viscous material from the fluid injector 100 and into the cannulated passage 16 of the bone screw 10. Additional connector or other quick coupling type components or mechanisms may additionally provided on the head portion 18 of the bone screw 10 in order to enable the injector tip 112 of the fluid injector to matingly couple with the injectable bone screw, and therefore permit the transfer of the viscous material from one to the other.

In another embodiment, the injector tip 112 of the fluid injector 100 is able to directly connect with the bone screw without requiring an adapter at all, or without a mating connection between the injector tip and the bone screw. For example, the injector tip 112 may seal directly to the top of the screw. In this case, no adapter is required to interconnect the fluid injector 110 and the bone screw 10, or at least no adapter opening in the screw body 12 is needed to permit the injector tip 112 to be connected to the screw 10, while still enabling the fluid injector 100 to inject the fluid therein into the cannulated passage 16 of the bone screw 10.

Once the screw 10 is inserted in the bone as depicted in FIG. 7A, the injector tip 112 of the fluid injector 100 can be connected in mating engagement with the adapter opening 24 in the exposed end of the bone screw 10, as shown in FIG. 7B. When the fluid injector 100 is connected to the screw 10, the fluid injector 100 can then be actuated to inject a viscous fluid into the bone and/or regions surrounding the bone screw 10, such as by depressing the plunger 109 of the syringe reservoir 108 (see FIG. 7A), thereby forcing the viscous fluid (such as the bone-treating fluid, for example) from the reservoir 108, through the needle portion 110 of the fluid injector 100 and into the cannulated passage 16 of the bone screw 10. The viscous fluid is then displaced through the fluid ejection passages 28 of the screw body 12 (such as through the interconnected pores therein, of otherwise), from the cannulated passage 16 to the bone surrounding the outer surface 26 of the screw 10. The viscous fluid is thus injected into the bone via the injected bone screw 10 located placed in situ with the insertion kit 400 within the bone B, the bone screw 10 therefore acting as an imbedded injection device for the fluid to be injected.

The above described methods are therefore used to position and insert a bone screw 10 in the scaphoid “S”, for example as depicted in FIGS. 7A and 7B, such that the bone screw 10 spans across the bone facture 120 in the scaphoid S. As the material or viscous fluid is forced into the bone screw 10 by the fluid injector 100, it is injected out through the cannulated passage 16 (see 7B) of the bone screw 10 and into the fracture crack 120 in the bone S. The combination of the fluid injector 100 and the bone screw 10 therefore together provide a fluid injection device kit which enables the injection of a viscous media directly into a bone or other tissue site from the inside thereof, which would be otherwise be difficult, at best, to access. In tests conducted in a fractured scaphoid bone S, where a gap was intentionally left to reproduce conditions where adjacent bone fragments are not perfectly in contact, a viscous media injected in the screw 10 was able to flow through the porosity of the material and flow in the gap 120 in the fracture of the scaphoid S.

It is to be understood that the material(s) chosen for the presently described bone screws is such that they are fully biocompatible and suitable for use in connection with fracture fixation within humans and animals.

The term “rigid” as used herein with reference to the foam material from which the present compression screws are formed is understood to mean structurally self-supporting and being sufficient strong (ex: has sufficient torsional stiffness) to withstand insertion into (and removal from) a bone element using an appropriate driving device (ex: screwdriver, powered or manual) without bending or substantially deflecting or compressing, etc.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure, and such modifications are intended to fall within the appended claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A system for inserting a bone screw into a tissue site comprising: a guide wire having a longitudinal axis and defining a non-circular cross-sectional shape in a plane substantially perpendicular to the longitudinal axis; and the bone screw including a screw body with at least one external thread thereon and having a central cannula extending longitudinally therethrough and configured for matingly receiving the guide wire therein, the cannula having a non-circular cross-section configured to rotationally couple the guide wire and the bone screw together such that relative axial rotation between the guide wire and the screw is prevented.

19. The system as defined in claim 18, wherein the non-circular cross-section of the cannula and the non-circular cross-sectional shape of the guide wire are the same.

20. The system as defined in claim 18, wherein the non-circular cross-section of the cannula is different from the non-circular cross-sectional shape of the guide wire, the cannula and the guide wire remaining rotationally coupled when mated together such that relative axial rotation between the guide wire and the screw is prevented.

21. The system as defined in claim 18, wherein the non-circular cross-sectional shape of at least one of the guide wire is at least one of hexagonal, octagonal, pentagonal, triangular, square, cross-shaped, star-shaped, oval, rectangular, or any combination thereof.

22. The system as defined in claim 18, wherein the guide wire is at least one of solid, hollow and perforated.

23. The system as defined in claim 18, further comprising a drive element configured to be removably engaged to the guide wire for rotation of the guide wire about the longitudinal axis thereof, thereby also rotating the screw when the screw and the guide wire are rotationally coupled together.

24. The system as defined in claim 23, wherein the drive element comprises a drive handle having a bore therein which corresponds to the non-circular cross-sectional shape of the guide wire, the guide wire being received within the bore of the drive handle such that the drive handle and the guide wire are rotationally coupled, whereby rotation of the drive handle will act to rotate the guide wire about the longitudinal axis thereof.

25. The system as defined in claim 18, wherein the screw body of the bone screw includes an annular portion which surrounds the cannula and which defines a radial wall thickness, the screw body comprising a number of fluid ejection passages therein which extend through the radial wall thickness between the cannula and an outer surface of the screw body, wherein the fluid ejection passages direct fluid in an outward direction from within the cannula to the outer surface of the screw body and into the tissue site surrounding the bone screw when the bone screw is inserted therein.

26. The system as defined in claim 25, wherein the screw body is composed at least partially of a rigid foam which defines a matrix defining a plurality of inter-connected pores therein, the plurality of interconnected pores being disposed throughout the radial wall thickness and defining said fluid ejection passages.

27. A method of inserting a bone screw into a tissue site, comprising:
positioning a proximal end of a guide wire into the tissue site at a desired location for insertion of the bone screw, the guide wire having a non-circular transverse cross-sectional shape and defining a longitudinal axis;
providing a cannulated bone screw having a non-circular cannula, and inserting the bone screw onto a distal end of the guide wire and sliding the bone screw longitudinally along the guide wire to the desired location, and ensuring that the bone screw and the guide wire are rotationally coupled such that relative rotation therebetween about the longitudinal axis is prevented;
engaging a drive handle to the guide wire such as to rotationally couple the drive handle and the guide wire; and
rotating the drive handle about the longitudinal axis such as to thereby rotate the guide wire and thereby rotate the bone screw which is rotationally coupled with the guide wire.

28. The method as defined in claim 27, wherein the step of engaging the drive handle to the guide wire further comprises inserting the drive handle onto the distal end of the guide wire and rotationally coupling the drive handle with the guide wire by mating a bore in the drive handle with the non-circular guide wire, the driving handle having a longitudinal axis corresponding to that of the guide wire

29. The method as defined in claim 27, wherein the non-circular cannula of the bone screw corresponds in cross-sectional shaped to the transverse cross-sectional shape of the guide wire, the step of rotationally coupling the bone screw and the guide wire including abutting corresponding surfaces of each of the non-circular cannula and the guide wire.

30. A bone screw comprising a screw body and having at least one external thread on an outer surface thereof, a cannulated passage extending through the screw body, and an inlet end of the screw body having an opening therein which opens into the cannulated passage and is configured to matingly receive therein an injector tip of a fluid injector for injecting a fluid into the cannulated passage of the bone screw, said cannulated passage having a non-circular cross-section configured to rotationally couple a guide wire having a non-circular cross-sectional shape such that relative axial rotation between the guide wire and the screw is prevented, the screw body having an annular portion which surrounds the cannulated passage therein and has a radial wall thickness, and a number of fluid flow passages extending through the radial wall and providing fluid flow communication between the cannulated passage and the outer surface of the screw body such as to direct fluid from within the cannulated passage in an outward direction through the screw body and into a tissue site surrounding the bone screw when the bone screw is inserted therein and the fluid is injected into the cannulated passage of the bone screw by the fluid injector.

31. The bone screw of claim 30, wherein the screw body is composed at least partially of a rigid foam which defines a matrix defining a plurality of inter-connected pores therein which are disposed throughout the radial wall thickness, the interconnected pores defining a plurality of said fluid flow passages.

32. The bone screw of claim 31, wherein the rigid foam is a porous sintered metal made from metal powders using powder metallurgy, the rigid metallic foam forming a metal matrix defining the pores throughout.

33. The bone screw of claim 32, wherein the rigid metallic foam comprises at least one of titanium, tantalum, magnesium, iron or an alloy of any one or more thereof.

34. The bone screw of claim 30, wherein the fluid flow passages have a cross-sectional size of less than 15 microns, of between 5 and 2000 microns, of between 10 and 1000 microns, of between 30 and 500 microns, or of between 50 and 400 microns which is sufficiently small to prevent bone ingrowth while still permitting fluid flow outwardly therethrough.

35. The bone screw of claim 30, wherein the bone screw is a head-less compression screw and the screw body includes at least a threaded leading end and a treaded trailing end, the threaded leading end having a pitch greater than that of the trailing end.

36. The bone screw of claim 30, wherein the opening in the inlet end of the bone screw is configured to engage an adapter used to fluidicly interconnect the injector tip of the fluid injector and the cannulated passage of the bone screw.

37. The bone screw of claim 36, wherein the opening in the inlet end of the bone screw has a greater diameter than the cannulated passage, the opening being sized to receive the adapter therein.

38. A kit for bone screw insertion within a tissue site comprising:
a guide wire having a longitudinal axis and defining a non-circular cross-sectional shape;
a bone screw having a screw body, at least one external thread on an outer surface thereof, and a cannulated passage extending longitudinally through the screw body, said cannulated passage having a non-circular cross-section configured to rotationally interconnect with the guide wire such that relative axial rotation between the guide wire and the bone screw is prevented, the screw body having an annular portion which surrounds the cannulated passage therein and has a radial wall thickness, the annular portion of the screw body having a number of fluid flow passages extending through the radial wall thickness to provide fluid flow communication between the cannulated passage and the outer surface of the screw body such as to direct a fluid within the cannulated passage through the screw body in an outward direction into the tissue site surrounding bone screw when the screw is inserted therein; and
a drive element which rotationally engages the guide wire for rotation of the guide wire about the longitudinal axis thereof, and thereby also rotating the bone screw when the bone screw and the guide wire are interconnected.

39. The kit as defined in claim 38, further comprising a fluid injector for injecting the fluid into the cannulated passage of the bone screw, the bone screw having an inlet end which includes an opening in communication with the cannulated passage and matingly receiving therein an injector tip of the fluid injector, the fluid injector including a storage reservoir for the bone growth promoting fluid in communication with the injector tip from which the fluid is ejected when the fluid injector is actuated.

40. The kit as defined in claim 39, wherein the fluid includes at least one of a bone repair promoting material and a bone-treating material.

41. The kit as defined in claim 40, wherein the bone repair promoting material includes a bone cement, and the bone-treating material includes at least one of an antibiotic and a chemotherapy agent.

42. The kit as defined in claim 38, wherein the screw body is composed at least partially of a rigid foam which defines a matrix defining a plurality of inter-connected pores therein which are disposed throughout the radial wall thickness, the interconnected pores defining a plurality of said fluid ejection passages.

43. The kit as defined in claim 42, wherein the rigid foam is a porous sintered metal made from metal powders using powder metallurgy, the rigid metallic foam forming a metal matrix defining the pores throughout, the rigid metallic foam being composed of at least one of titanium, tantalum, magnesium, iron or an alloy of any one or more thereof.

44. The kit as defined in claim 38, wherein the opening of the cannulated passage defines an adapter opening which has a greater diameter than the cannulated passage, the adapter opening being sized to receive a adapter therein, the adaptor linking the bone screw and the injector tip of the fluid injector.
